(19) United States
(12) Patent Application Publication
(10) Pub. No.: US 2007/0135791 A1
(43) Pub. Date: Jun. 14, 2007

(54) Method for Infusing the Interior of a Blood Vessel

(76) Inventors: Charles R. Slater, Fort Lauderdale, FL (US); Brett E. Naglreiter, Austin, TX (US); Scott L. Jahrmarkt, Miami Beach, FL (US); Banning G. Lary, Miami, FL (US)

Correspondence Address: GORDON & JACOBSON, P.C., 60 LONG RIDGE ROAD, SUITE 407, STAMFORD, CT, 06902, US

(21) Appl. No.: 11/624,412

(22) Filed: Jan. 18, 2007

Related U.S. Application Data
(63) Continuation-in-part of application No. 10/922,221, filed on Aug. 19, 2004.
(63) Continuation-in-part of application No. 10/922,123, filed on Aug. 19, 2004.
(63) Continuation-in-part of application No. PCT/US06/01458, filed on Jan. 13, 2006.

(51) Int. Cl. A61M 31/00

(52) U.S. Cl. 604/508; 604/509

(57) ABSTRACT

Methods for infusing the interior of a blood vessel are practiced with a catheter having an infusion lumen, a plurality of elution holes, a movable barrier (preferably an inflatable bladder) between the infusion lumen and the elution holes, an occlusion balloon, and an inflation lumen. The methods include sealing the elution holes with the movable barrier, inserting the catheter into the blood vessel, inflating the occlusion balloon, unsealing the elution holes, injecting the therapeutic agent through the infusion lumen, deflating the occlusion balloon and removing the catheter from the blood vessel. The methods also preferably include testing the occlusion balloon and priming the infusion lumen prior to sealing the infusion ports and inserting the catheter into the blood vessel.

### CROSS-REFERENCE TO RELATED APPLICATIONS

This application is a continuation-in-part of applications Ser. Nos. 10/922,221 and 10/922,123 both filed on Aug. 19, 2004 and application PCT/US2006/001458 filed Jan. 13, 2006, the contents of which are hereby incorporated herein by reference. This application is also related to co-pending application—[VRX-007] filed simultaneously herewith, the contents of which are also hereby incorporated herein by reference.

### BACKGROUND OF THE INVENTION

1. Field of the Invention

The invention relates to the treatment and correction of venous insufficiency. More particularly the invention relates to a minimally invasive procedure using a catheter-based system to treat the interior of a blood vessel. The invention has particular application to varicose veins although it is not limited thereto.

2. State of the Art

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sac or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

Incompetence in the venous system can result from vein dilation. Separation of the cusps of the venous valve at the commissure may occur as a result. Two venous diseases which often involve vein dilation are varicose veins and chronic venous insufficiency.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

Phlebosclerosis, the destruction of venous channels by the injection of sclerosing agents, has been used to treat varicose veins since 1853, when Cassaignae and Ebout used ferric chloride. Sodium salicylate, quinine, urea, and sodium chloride have also been used, but the agent more recently favored is sodium tetradecyl sulfate. In order for phlebosclerosis to be effective, it is necessary to evenly dispense the sclerosing agent throughout the wall of the vein without using toxic levels of the sclerosing agent. This is not particularly difficult for the smaller veins. However, it is quite difficult or nearly impossible in larger veins. When a larger vein is injected with a sclerosing agent, the sclerosing agent is quickly diluted by the substantially larger volume of blood which is not present in smaller veins. The result is that the vein is sclerosed (injured) only in the vicinity of the injection. If the procedure is continued, and the injections are far apart, the vein often assumes a configuration resembling sausage links. The problem cannot be cured by injecting a more potent solution of sclerosing agent, because the sclerosing agent may become toxic at such a concentration.

U.S. Pat. No. 5,676,962 discloses an injectable micro foam containing a sclerosing agent. The microfoam is injected into a vein where it expands and, theoretically, achieves the same results as a larger quantity of sclerosing agent without the toxicity. Such foam is presently manufactured under the trademark Varisolve® by Provensis, Ltd., London, England. Recent clinical trials of the foam indicate a success rate of 81%.

Until recently, the preferred procedure for treating the great saphenous vein was surgical stripping. This highly invasive procedure involves making a 2.5 cm incision in the groin to expose the saphenofemoral junction, where the great saphenous vein and its branches are doubly ligated en masse with a heavy ligature. The distal portion of the vein is exposed through a 1-cm incision anterior to the medial malleolus, and a flat metal or plastic stripper is introduced to exit in the proximal saphenous vein. The leg is held vertically for 30 seconds to empty the venous tree before stripping the vein from the ankle to the groin. If the small saphenous vein is also incompetent, it is stripped at the same time from an incision posterior to the lateral malleolus to the popliteal space. After stripping the veins, the leg is held in the vertical position for three to four minutes to permit broken vessel ends to retract, constrict, and clot.

After the stripping procedure, collateral veins are removed by the avulsion-extraction technique. By working through small (5 to 8 mm) transverse incisions, segments of vein 10 to 20 cm long can be removed by dissecting subcutaneously along the vein with a hemostat, and then grasping, avulsing, and removing the vein. With practice, long segments of vein in all quadrants can be removed through these small incisions. No attempt is made to ligate the branches or ends of the veins, since stripping has shown it to be unnecessary. Bleeding is controlled by elevation and pressure for two to four minutes. As many as 40 incisions are made in severe cases, but their small size and transverse direction permit closure with a single suture.

Before closure of the incisions, a rolled towel is rolled repeatedly from the knee to the ankle and from the knee to the groin to express any clots that may have accumulated. The groin incision is approximated with three 5-0 nylon mattress sutures and all other incisions are closed with a single suture.

As can be readily appreciated, the stripping and avulsion-extraction procedures are relatively invasive and require significant anesthesia. It can therefore be appreciated that it would be desirable to provide an alternative, less invasive procedure which would accomplish the same results as stripping and avulsion-extraction.

Recently, a number of patents have issued disclosing the treatment of varicose veins with RF energy. Illustrative of these recent patents are: U.S. Pat. No. 6,200,312 entitled “Expandable Vein Ligator Catheter Having Multiple Electrode Leads”; U.S. Pat. No. 6,179,832 entitled “Expandable Catheter Having Two Sets of Electrodes”; U.S. Pat. No. 6,165,172 entitled “Expandable Vein Ligator Catheter and Method of Use”; U.S. Pat. No. 6,152,899 entitled “Expandable Catheter Having Improved Electrode Design, and Method for Applying Energy”; U.S. Pat. No. 6,071,277 entitled “Method and Apparatus for Reducing the Size of a Hollow Anatomical Structure”; U.S. Pat. No. 6,036,687 entitled “Method and Apparatus for Treating Venous Insufficiency”; U.S. Pat. No. 6,033,398 entitled “Method and Apparatus for Treating Venous Insufficiency Using Directionally Applied Energy”; U.S. Pat. No. 6,014,589 entitled “Catheter Having Expandable Electrodes and Adjustable Stent”; U.S. Pat. No. 5,810,847 entitled “Method and Apparatus for Minimally Invasive Treatment of Chronic Venous Insufficiency”; U.S. Pat. No. 5,730,136 entitled “Venous Pump Efficiency Test System And Method”; and U.S. Pat. No. 5,609,598 entitled “Method and Apparatus for Minimally Invasive Treatment of Chronic Venous Insufficiency”. These patents generally disclose a catheter having an electrode tip which is switchably coupled to a source of RF energy. The catheter is positioned within the vein to be treated, and the electrodes on the catheter are moved toward one side of the vein. RF energy is applied to cause localized heating and corresponding shrinkage of the adjacent venous tissue. After treating one section of the vein, the catheter can be repositioned to place the electrodes to treat different sections of the vein.

Although this procedure has gained acceptance and is less invasive than the stripping and avulsion-extraction procedures, there are several disadvantages to it. In particular, RF treatment is actually quite slow and painful and the patient must be sufficiently anaesthetized along the entire length of the veins to be treated. In addition, repositioning the catheter is time consuming thus requiring anesthesia for a prolonged period. Moreover, the RF treatment is incomplete, as only a portion of the vein wall is actually treated, i.e. the portion contacting the electrode. The partially treated vein may eventually recanalize. Furthermore, tributary veins remain unaffected and must be treated separately. In addition, for even and consistent cauterization, RF treatment requires that the practitioner be keenly aware of the procedure time. If RF energy is applied for too long, it can cause undesired burns. If RF energy is not applied long enough, the treatment is ineffective.

In addition to RF treatment, laser treatment has been used with some success. Laser treatment shares many of the disadvantages of RF treatment. In particular, as with the RF devices, the practitioner must be very careful as to the intensity and duration of the treatment to assure that the treatment is effective but without causing undesired burns.

3. Parent Applications

The parent applications disclose apparatus and methods of introducing a therapeutic agent into a vein. One method comprises introducing a catheter into the vein, the catheter having a plurality of infusion ports and an infusion lumen, activating an occlusion device on the catheter to occlude blood flow within the vein, removing a barrier from at least one of the plurality of infusion ports and infusing a therapeutic agent from the infusion lumen through the ports and into the vein. The introducing step may comprise introducing the catheter into the saphenous vein. Introducing the catheter into the saphenous vein may occur in the vicinity of the knee or the vicinity of the ankle. The activation of the occlusion device may comprise inflating an occlusion balloon and/or isolating the saphenofemoral junction from the infusion ports. The step of removing a barrier may comprise deflating an elongate tubular bladder. The method may further comprise enhancing drainage of the vein by raising the position of the vein relative to the location of the occlusion device. The method may also comprise lowering the position of the vein relative to the location of the occlusion device to facilitate migration of the therapeutic agent along the vein wherein the therapeutic agent is a foam. The method may also comprise maintaining a raised position of the vein relative to the location of the occlusion device to facilitate migration of the therapeutic agent to the saphenofemoral junction.

In another embodiment, a method of inhibiting retrograde flow of body fluid through effluent ports and into the infusion lumen of a catheter is provided. The method comprises the steps of providing a fluid delivery catheter, having an elongate body, at least one effluent port on the body and an infusion lumen extending within the body, inflating a flow regulator within the tubular body to isolate the effluent port from the infusion lumen and introducing the catheter into a patient in a location that exposes the catheter to a body fluid wherein the flow regulator inhibits retrograde flow of body fluid through the effluent port and into the infusion lumen. The step of inflating a flow regulator may comprise inflating an elongate tubular balloon. The method may additionally comprise the step of deflating the flow regulator to place the effluent port in communication with the infusion lumen.

The apparatus described in the parent applications includes a catheter assembly having a proximal hub with three valves: an occlusion balloon inflation valve, an infusion valve, and a bladder valve. The infusion valve is preferably a check valve which assists in maintaining the primed infusion lumen during preparation before the procedure.

### SUMMARY OF THE INVENTION

It is therefore an object of the invention to provide methods for infusing the interior of a blood vessel with a therapeutic agent.

In accord with this object, which will be discussed in detail below, the methods of the invention are practiced with a catheter having an infusion lumen, a plurality of elution holes, a movable barrier (preferably an inflatable bladder) between the infusion lumen and the elution holes, a blood vessel occluder (preferably an occlusion balloon), and an inflation lumen. The methods include sealing the elution holes with the movable barrier, inserting the catheter into the blood vessel, inflating the occlusion balloon, unsealing the elution holes, injecting the therapeutic agent (preferably a sclerosing foam) through the infusion lumen, deflating the occlusion balloon and removing the catheter from the blood vessel. The methods also preferably include testing the occlusion balloon and priming the infusion lumen prior to sealing the elution holes and inserting the catheter into the blood vessel. The balloon is preferably tested by purging air from the balloon, inflating it with saline or contrast media and inspecting it for leaks, then deflating it. The infusion lumen is preferably primed by unsealing the elution holes and injecting the therapeutic agent until it flows through all of the elution holes.

The step of inserting the catheter into the blood vessel is preferably preceded by inserting a sheath introducer into the blood vessel. The step of inserting also preferably includes locating the occlusion balloon under ultrasonic or fluoroscopic guidance before inflating it. Those skilled in the art will also appreciate that after the catheter is removed from the blood vessel, the sheath is also removed.

Additional objects and advantages of the invention will become apparent to those skilled in the art upon reference to the detailed description taken in conjunction with the provided figures.

### BRIEF DESCRIPTION OF THE DRAWINGS

FIG. 1 is a broken longitudinal side elevation view of a catheter apparatus used to perform the methods of the invention;

FIG. 2 is a cross section of the catheter taken along line 2-2 in FIG. 1 with the sealing bladder deflated;

FIG. 3 is a view similar to FIG. 2 but with the sealing bladder inflated;

FIG. 4 is a cross section taken along line 4-4 in FIG. 2;

FIG. 5 is a cross section taken along line 5-5 in FIG. 3; and

FIGS. 6-14 are schematic views illustrating use of the device in performing the methods of the invention.

### DETAILED DESCRIPTION OF THE PREFERRED EMBODIMENTS

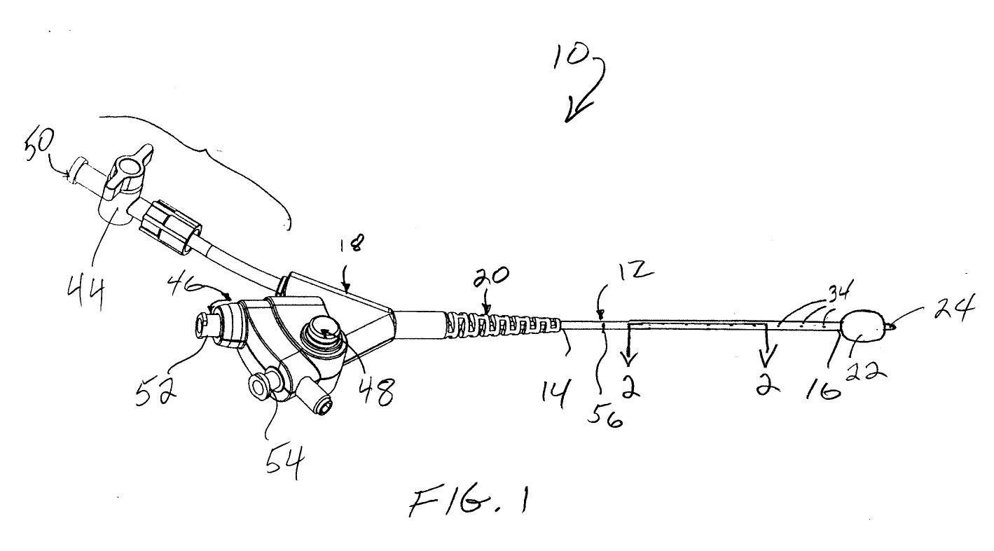

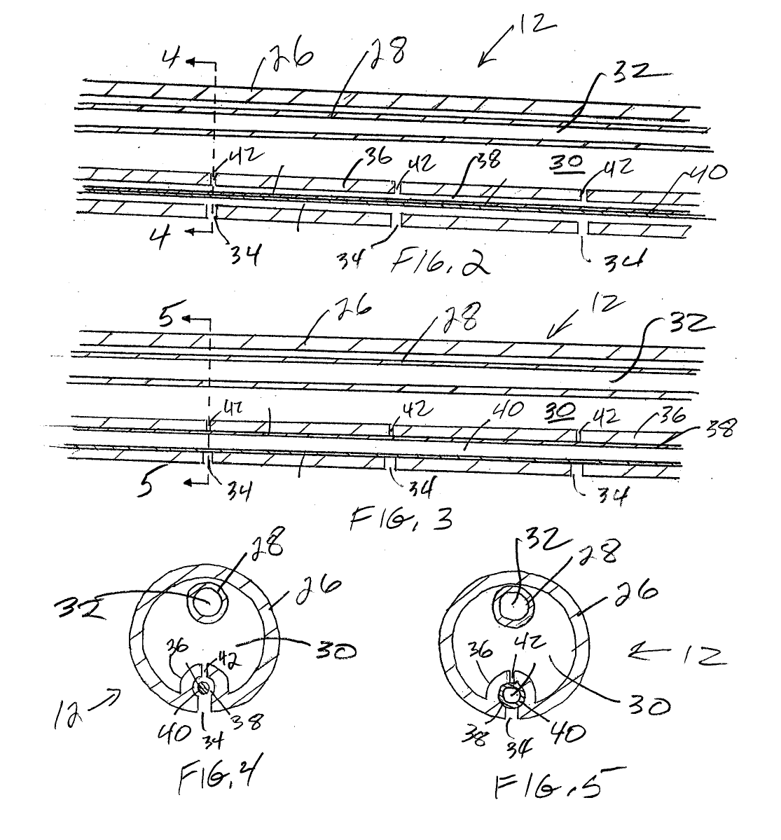

Turning now to FIG. 1, an infusion catheter apparatus 10 suitable for practicing the methods of the invention includes a catheter assembly 12 having a proximal end 14 and a distal end 16. The proximal end 14 is coupled to a valve body 18 via a strain relief 20. The distal end 16 is coupled to an occlusion balloon which has an atraumatic tip 24. As seen best in FIGS. 2-5, the catheter assembly 12 includes outer catheter tube 26 and an inner catheter tube 28. The outer catheter tube 26 defines an infusion lumen 30 and the inner catheter tube 28 defines an inflation lumen 32. The outer tube 26 is also provided with a plurality of elution holes 34 which extend along a length of the outer tube 26. An interior tangential tube 36 defines a side lumen 38 which contains an inflatable bladder tube 40. The tube 36 is provided with a plurality of infusion holes 42 along at least a portion of its length. When the bladder tube 40 is deflated as shown in FIGS. 2 and 4, the infusion lumen 30 is in fluid communication with the side lumen 38 via the infusion holes 42 and the side lumen 38 is in fluid communication with the elution holes 34. When the bladder tube is inflated as shown in FIGS. 3 and 5, fluid communication from the infusion lumen into the side lumen 38 and out through the elution holes 34 is blocked.

Returning now to FIG. 1, the valve body 18 includes three valves: an occlusion balloon inflation valve 44, an infusion check valve 46, and a bladder tube pressure release valve 48. The balloon inflation valve is coupled to a balloon inflation luer 50 and to the lumen 32 of the inner catheter tube 28 (FIGS. 2-5) which is coupled to the balloon 22. The infusion check valve 46 is coupled to an infusion luer 52 and to the lumen 30 of the outer catheter tube 26. The bladder tube pressure release valve 48 is coupled to a bladder tube inflation luer 54 and to the bladder tube 40. The valve 48 is preferably a releasable check valve so that the bladder tube can be inflated without manipulating the valve and can be deflated by pushing down on the valve button. According to the presently preferred embodiment the catheter assembly 12 has a proximal marker 56 which is used to determine whether the catheter assembly is sufficiently inserted into the blood vessel such that the elution holes 34 are all located inside the blood vessel. As mentioned above and described in detail below with reference to FIGS. 12-14, the assembly 12 is delivered to the treatment location through an introducer sheath and if the assembly is not inserted far enough into the sheath, the sheath could block some of the elution holes 34. According to one embodiment of the invention the overall length of the entire apparatus is approximately 83 cm. The distance between the most proximal elution hole and the marker 56 is approximately 12 cm. According to this embodiment, nine different models are provided where the number of elution holes can be as few as six or as many as twenty-two. In the case of six elution holes, the “infusion length” is approximately 12 cm. In the case of twenty-two elution holes, the infusion length is approximately 44 cm.

From the foregoing, those skilled in the art will appreciate that when the valve 44 is opened and a syringe (not shown) filled with saline or a contrast mixture is attached to the luer 50, injection will cause the occlusion balloon 22 to inflate. Closing the valve 44 will keep the balloon 22 inflated even after the syringe is removed. Similarly, when a syringe filled with saline is attached to the luer 54, injection will cause the bladder tube 40 to inflate. Also, when a syringe filled with treating agent is coupled to the luer 52, injection will cause the agent to enter the infusion lumen 30 and, if the bladder tube 40 is deflated, exit the elution holes 34.

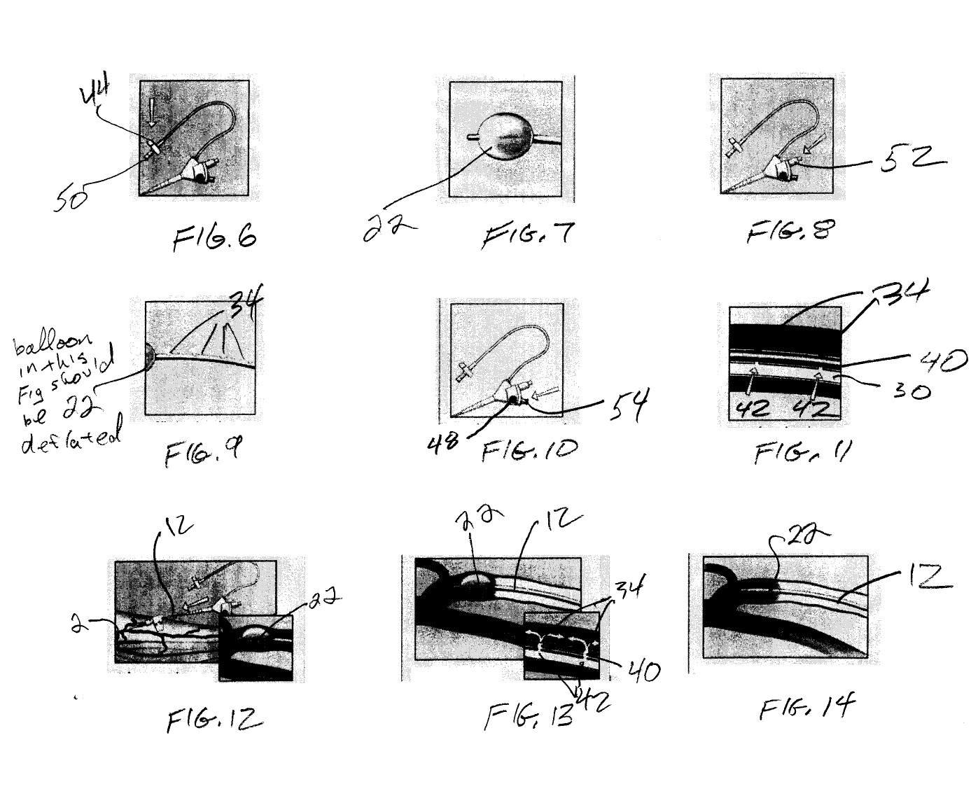

Referring now to all of the Figures generally and FIGS. 6-14 in sequence, the preferred methods of the invention will be explained. The first step is to purge the occlusion balloon 22 of air and to see if it leaks. This is done by opening the valve 44, attaching a syringe (not shown) to the luer 50, and sucking air out of the balloon with the syringe, then closing the valve 44. With the balloon purged and the valve closed, a syringe (not shown) containing saline or a contrast mixture is attached to the luer 50. The valve 44 is opened and the liquid is injected into the balloon. The amount of liquid used to inflate the balloon depends on the diameter of the blood vessel to be treated. It can be as little as 0.2 ml for a 3.0 mm diameter blood vessel to as much as 7.7 ml for a 19 mm diameter blood vessel. After the balloon is tested as illustrated in FIGS. 6 and 7, and prior to insertion of the catheter into the blood vessel, the balloon is deflated and the valve 44 is closed. Also prior to insertion, a 10 ml or 20 ml syringe (not shown) filled with at least 5 ml of therapeutic agent is attached to the luer 52 (FIG. 8). Agent is injected until it flows through all of the elution holes 34 as shown in FIG. 9. With the infusion lumen 30 primed with agent, the bladder tube 40 is inflated. This is accomplished by attaching a 1 ml syringe (not shown) to the luer 54 and injecting 0.75 ml of sterile saline. The valve 48 keeps the bladder tube 40 inflated blocking the holes 34 and 42 as shown in FIGS. 10 and 11.

After the foregoing preparation, the blood vessel may be treated as follows with reference to FIGS. 12-14. A sheath introducer 1 is first inserted into the blood vessel 2 to be treated. The catheter assembly 12 is advanced through the sheath introducer 1 into the blood vessel 2 until the proximal marker 56 is not visible and until the tip of the catheter reaches a desired location. The location of the catheter assembly 12 and the occlusion balloon 22 is confirmed with ultrasound or fluoroscopy. The occlusion balloon 22 is then inflated until desired occlusion and stability is achieved. With the catheter assembly 12 and the occlusion balloon in position as shown in FIGS. 12 and 13, the bladder tube 40 is deflated by pressing the valve button 48 (FIG. 1) and treating agent is injected through the check valve 46. After treatment, the occlusion balloon 22 is deflated as shown in FIG. 14 by opening the valve 44 (FIG. 1) and the apparatus is removed through the sheath introducer 1. The sheath introducer 1 is then removed from the blood vessel 2 and homeostasis is achieved.

As mentioned above, the methods of the invention are particularly well suited for the treatment of varicose veins and in particular the great saphenous vein. More particularly, preferably, a patient is first evaluated to determine the length and volume of the vein to be treated so that a catheter of appropriate length with an occlusion balloon of appropriate size can be selected. This evaluation typically occurs prior to the day of the procedure but could occur on the same day. If the procedure is performed at a later date, the initial evaluation is preferably confirmed. A quantity of foam sclerosant is selected based on the calculated volume of the vein to be treated. Methods for calculating the volume of the vein are disclosed in previously incorporated [VRX-007]. The preparation of the catheter is performed as described above. The vein is accessed with a 7 French introducer and the tip of the catheter is advanced through the vein to the proximal-most treatment point. Treatment proceeds as described above with the patient's leg remaining supine with the catheter in place for four minutes before the occlusion balloon is deflated and the catheter is removed. When the catheter is removed, pressure is applied to the access site and the leg is wrapped with gauze to minimize irritation. A six inch long feminine pad is placed along the great saphenous vein starting from the proximal treatment point and an STD [WHAT IS STD?] foam pad is placed distal to the feminine pad for the remaining treated length of the vein. The pads are then wrapped with a self-adherent elastic wrap such as 3M COBAN wrap. The treated leg is then placed in a thigh high compression (30-40 mm Hg) stocking such as the SIGVARIS stockings from Ganzoni & Cie, St. Gallen, Switzerland. The patient is then instructed to ambulate for a minimum of 15 minutes. A first follow-up examination is performed 48 hours later at which time the pads and the elastic wrap are removed. The patient continues to wear the stocking(s) 24 hours per day for the next week and during daytime only for two more weeks. Additional examinations of the patient are performed at 1 month, 3 months, 6 months, 12 months, 18 months and 24 months. During each examination an ultrasound assessment of the deep venous system, treated vein, tributaries and perforators is preformed.

The catheter ensures even and simultaneous infusion of foam. The occlusion balloon isolates the treatment area and minimizes drug dilution. The bladder tube guarantees complete infusion control and maintains the primed catheter during pre-op. This substantially pain-free procedure eliminates tumescent anesthesia. The treatment extends into incompetent tributaries. The methods are easily learned by practitioners and the entire procedure can be performed in as little as twenty minutes. The use of a foam sclerosant increases drug contact area, displaces blood through the low density of the foam, enables echogenicity of the drug with ultrasound and requires 80% less dosage than a liquid sclerosant.

There have been described and illustrated herein a method for infusing the interior of a blood vessel. While a particular embodiment of the invention has been described, it is not intended that the invention be limited thereto, as it is intended that the invention be as broad in scope as the art will allow and that the specification be read likewise. Thus, while a particular apparatus for performing the method has been disclosed, it will be appreciated that other apparatus could be used as well. Several such apparatus are disclosed in the previously incorporated parent applications. In addition, while every method step has been disclosed in a particular order, it will be understood that some of the method steps can be performed in different order and that some of the method steps, while desirable, are not absolutely necessary. It will therefore be appreciated by those skilled in the art that yet other modifications could be made to the provided invention without deviating from its spirit and scope as claimed.

What is claimed is:

1. A method of introducing a therapeutic agent into a blood vessel using a catheter having a blood vessel occluder and a plurality of sealable elution holes, said method comprising:
sealing the elution holes;
delivering the catheter into the blood vessel;
activating the occluder to occlude the blood vessel;
unsealing the elution holes;
injecting the therapeutic agent into the catheter and out of the elution holes into the blood vessel;
deactivating the occluder; and
removing the catheter from the blood vessel.

2. The method according to claim 1, further comprising:
prior to sealing the elution holes, priming the catheter with the therapeutic agent.

3. The method according to claim 1, further comprising:
prior to delivering the catheter into the blood vessel, testing the occluder by activating and deactivating it.

4. The method according to claim 1, wherein:
said step of sealing the elution holes includes inflating a bladder tube.

5. The method according to claim 4, wherein:
said step of unsealing the elution holes includes deflating the bladder tube.

6. The method according to claim 1, wherein:
said step of activating the occluder includes inflating a balloon.

7. The method according to claim 6, wherein:
said step of deactivating the occluder includes deflating the balloon.

8. The method according to claim 1, further comprising:
prior to said step of delivering the catheter into the blood vessel, inserting a sheath introducer into the blood vessel, wherein
said step of delivering the catheter into the blood vessel includes delivering the catheter through the sheath introducer.

9. The method according to claim 8, further comprising:
after said step of removing the catheter from the blood vessel, removing the sheath introducer from the blood vessel.

10. The method according to claim 1, wherein:
the catheter includes a proximal marking and said step of delivering the catheter into the blood vessel includes inserting the catheter into the blood vessel until the proximal marking cannot be seen.

11. A method for treating an incompetent blood vessel with a sclerosant foam using a catheter having a blood vessel occluder and a plurality of sealable elution holes, said method comprising:
sealing the elution holes;
delivering the catheter into the blood vessel;
activating the occluder to occlude the blood vessel;
unsealing the elution holes;
injecting the sclerosant foam into the catheter and out of the elution holes into the blood vessel;
deactivating the occluder; and
removing the catheter from the blood vessel.

12. The method according to claim 11, further comprising:
prior to sealing the elution holes, evaluating the blood vessel to be treated and selecting a catheter of appropriate length and occluder of appropriate size.

13. The method according to claim 11, further comprising:
prior to injecting the scierosant foam into the catheter and out of the elution holes into the blood vessel, calculating the volume of sclerosant foam to be injected based on the volume of the blood vessel being treated.

14. The method according to claim 11, further comprising:
prior to delivering the catheter into the blood vessel, accessing the blood vessel with a 7 French introducer.

15. The method according to claim 11, further comprising:
after injecting the sclerosant foam, waiting at least four minutes before deactivating the occluder.

16. The method according to claim 11, further comprising:
after removing the catheter, applying pressure to treated blood vessel.
